(19) United States
(12) Patent Application Publication
(10) Pub. No.: US 2011/0192399 A1
(43) Pub. Date: Aug. 11, 2011

(54) Vaporizer with improved heating chamber and related methods

(76) Inventors: Lance Alexander Wilke, Chico, CA (US); Sean David Crawford, Chico, CA (US)

(21) Appl. No.: 12/658,544

(22) Filed: Feb. 11, 2010

(51) Int. Cl. A61M 11/00

(52) U.S. Cl. 128/203.27; 392/390

(57) ABSTRACT

In one embodiment, a vaporizer for vaporizing a substance is described herein. The vaporizing includes a vapor chamber; a heating chamber; and a cartridge positioned between the vapor chamber and the heating chamber. The vapor chamber, the cartridge, and the heating chamber form a continuous bidirectional pathway for airflow within the vaporizer. The cartridge is configured to contain the substance to be vaporized, and the heating chamber includes a heating core having a heating element disposed therein for convection heating of a portion of the airflow that vaporizes the substance contained in cartridge.

### TECHNICAL FIELD

The disclosure herein relates to a vaporizer and related methods, and more particularly to a vaporizer having an improved heating chamber for convection type heating of air used during, among other things, heating of a substance for inhalation.

### BACKGROUND

Vaporizers also known as a humidifier are common place and are generally utilized for the purpose of aroma and/or inhalation therapy. In this regard, vaporizers heat a substance, herbs for example, such as tobacco, cannabis, lavender, chamomile, and many other types of plant material.

The vaporizer may work by heating the substance through the use of direct heat or the use of hot air in one of three ways. The first is thermal conduction where the substance is set directly on a heating element such as a metal plate. The second is thermal radiation in which light is used to heat the substance. The third is convection where the substance never touches the heating element; instead hot air is passed over the substance.

At lower levels of heat, vapors extracted from vegetable materials are mainly aroma therapeutic (inactive fragrance) and do not usually contain the active ingredients of the substance. Without the active ingredients being present, there is no physiological reaction. At higher levels of heat, active ingredients will be included in the vapor given off during heating. Usually aromatic vapors have already been released and are not always present at the higher heat levels. With some substances, such as cannabis, active ingredients appear at different levels of heat.

After the substance is heated a mist or vapor containing some aspect of the substance is released and either enjoyed as an aromatic or inhaled to obtain a physiological reaction. The warm air containing the substance product can be harsh on the throat and bronchial tubes. Accordingly, some vaporizer use a cooling down process that allows water moisture to be included in the vapor produced. These vaporizers enable the user to inhale a cool moist vapor that is relatively less harsh and irritating.

Vaporizers are favored over traditional methods of heating or smoking substances due to the reduction of harsh side effects. Some of these side effects include inhalation of tar, carbon monoxide, and other carcinogens either directly or from second hand smoke. With many states imposing smoking bans in public areas, vaporizers have become popular substitutes.

As indicated above, vaporizers work by heating a substance to extract a product of the substance. Such heat may be facilitated by the use of butane, open flame, or electricity. Although generally well suited for their intended purpose, the vaporizer described herein has a heating chamber configured and designed to improve heating efficiency and reduce the heating time of the heating element prior to use when compared with a similar type vaporizer for convection type heating of air used during, among other things, heating of a substance for inhalation.

### SUMMARY

For the purpose of summarizing the invention certain objects and advantages have been described. it is to be understood that not all such objects or advantages may be achieved in accordance with any particular embodiment. Thus, for example, those skilled in the art will recognize that the device described herein may be embodied or carried out in a manner that achieves or optimizes one advantage or group of advantages as taught herein without necessarily achieving other objects or advantages.

In one embodiment the disclosed subject matter includes a vaporizer for vaporizing a substance, the vaporizing including a vapor chamber; a heating chamber; and a cartridge positioned between the vapor chamber and the heating chamber. The vapor chamber, the cartridge, and the heating chamber form a continuous bidirectional pathway for airflow within the vaporizer. The cartridge is configured to contain the substance to be vaporized, and the heating chamber includes a heating core having a heating element disposed therein for convection heating of a portion of the airflow that vaporizes the substance contained in cartridge.

In another embodiment the disclosed subject matter includes a heating chamber for heating a portion of airflow within a vaporizer. The heating chamber includes a heating core formed in the center of the heating chamber, and a heating element disposed within the heating core for convection heating of a portion of the output airflow within the vaporizer.

These and other embodiments will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIGS. 1A-1C are perspective views of one embodiment showing a case for a vaporizer.

FIG. 2A is a see-through view of one embodiment of the vaporizer showing a portion of the central cavity.

FIG. 2B is a cut-away view of one embodiment of the vaporizer showing a power source, a wiring and circuitry pack, a heating chamber and core for containing a heating element such as a coil, and the central cavity, as well as bidirectional flow of air within the vaporizer.

FIG. 3 is a cut-away view of one embodiment of the heating chamber, a cartridge, and the lower portion of a vapor chamber showing bidirectional flow of air within the vaporizer.

FIGS. 4A and 4B show perspective views of the heating chamber, the core, and the heating element of the vaporizer.

FIG. 5 is a perspective view of a cartridge of the vaporizer.

### DETAILED DESCRIPTION

Exemplary embodiments will now be described with references to the accompanying figures, with like reference numerals referring to like elements throughout. The terminology used in the description is not intended to be interpreted in any limited or restrictive manner, simply because it is being utilized in conjunction with a detailed description of certain embodiments. Furthermore, various embodiments (whether or not specifically described herein) may include novel features, no single one of which is solely responsible for its desirable attributes or which is essential to practicing the subject matter described herein.

As indicated above, a vaporizer may work by heating the substance to extract a product of the substance in vapor form that has the appearance of light smoke. The content of the vapor is different than smoke in that the vapor ideally contains zero particulate matter (tar) and reduced noxious gases such as carbon monoxide. Vapors may be filtered and cooled further by various methods. The vapors may then be inhaled directly or stored for subsequent use. With little or no smoke produced, cooler temperatures, and less material required to achieve a comparable effect as with more traditional methods, the irritating/harmful effects of smoking are greatly reduced or eliminated along with second hand smoke by using a vaporizer. As no combustion occurs, fewer chemical changes occur during vaporization making vaporizers useful in many aspects including use in places where there are public bans on smoking.

The subject matter described herein relates to a vaporizer having an improved heating chamber for convection type heating of air used during, among other things, heating of a substance for inhalation. The vaporizer is configured and designed to improve heating efficiency and reduce heating time of the heating element or coil when compared with a similar type vaporizer.

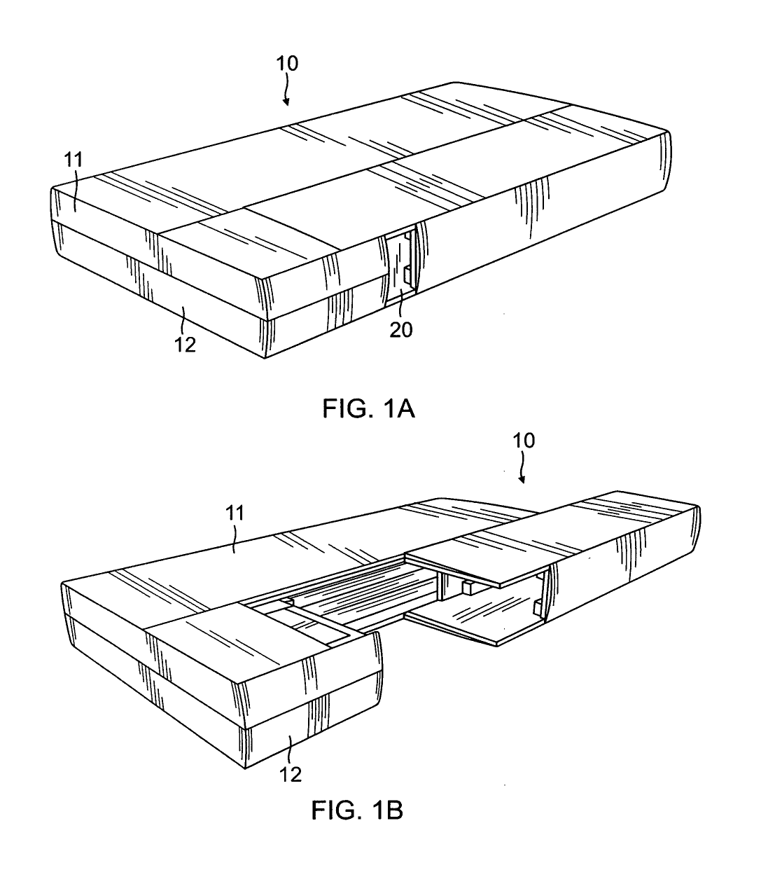

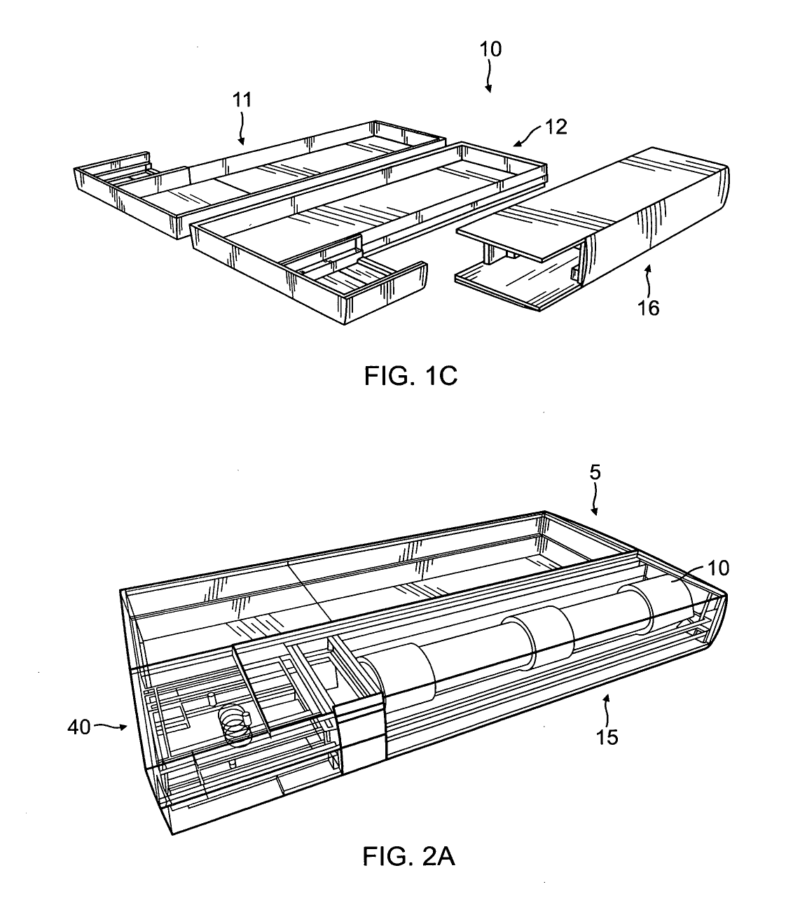

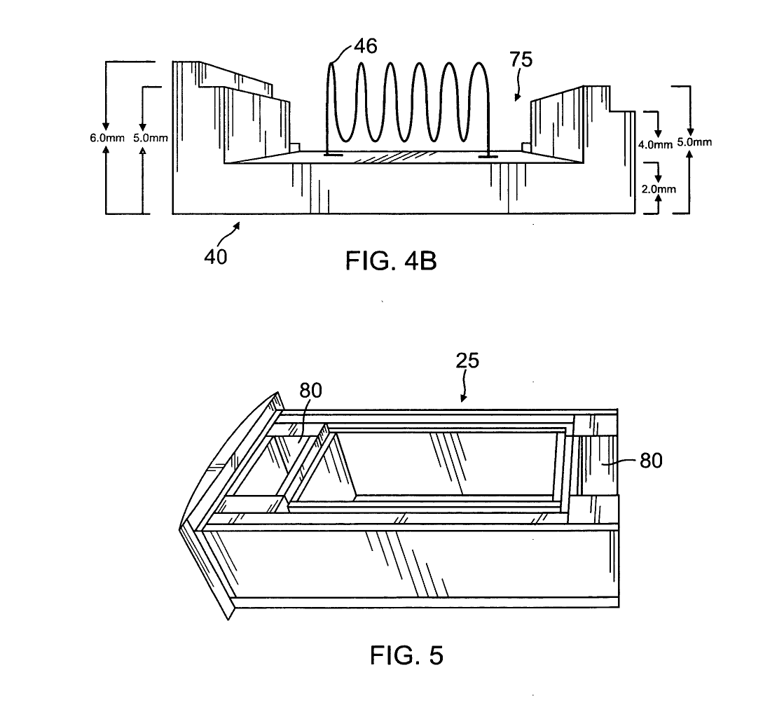

FIGS. 1A-1C and FIGS. 2A and 2B are perspective views showing one embodiment of the vaporizer. The vaporizer 5 may be configured to include a case 10 for housing various components of the vaporizer 5 as described in more detail below. The case 10 may include a first sidewall portion 11 and second sidewall portion 12 that are mirror images of each other. The case 10 may be constructed of a plastic, polycarbonate, or similar type durable material and the first sidewall portion 11 and the second sidewall portion 12 may be affixed together with an epoxy or other suitable adhesive type product. The first sidewall portion 11 and the second sidewall portion 12 may be compartmentalized to enclose components of the vaporizer 5 including a power source 30 such as a battery pack 31, wiring and circuitry 35, and a heating chamber 40. The case 10 may further include a removable elongated vapor chamber 15 and associated vapor chamber case 16 that is fitted to the first sidewall portion 11 and the second sidewall portion 12 by means of a tongue and groove mating mechanism to facilitate cleaning and replacement. A space or opening 20 may be provided between the mating surfaces of the first sidewall portion 11, the second sidewall portion 12, and the removable vapor chamber case 16 for insertion and removal of a cartridge 25 (see FIG. 5) that is configured to hold a substance such as plant matter that is to be heated by hot air.

As shown more specifically in FIGS. 2A and 2B, the vaporizer 5 includes a power source 30 such as a battery pack 31 and associated circuitry 35 for supplying power to a heating element 45 such as a coil 46 positioned within a heating core 75 of the heating chamber 40. In one embodiment the battery pack 31 includes two rechargeable 3.7 volt lithium polymer batteries wired in series.

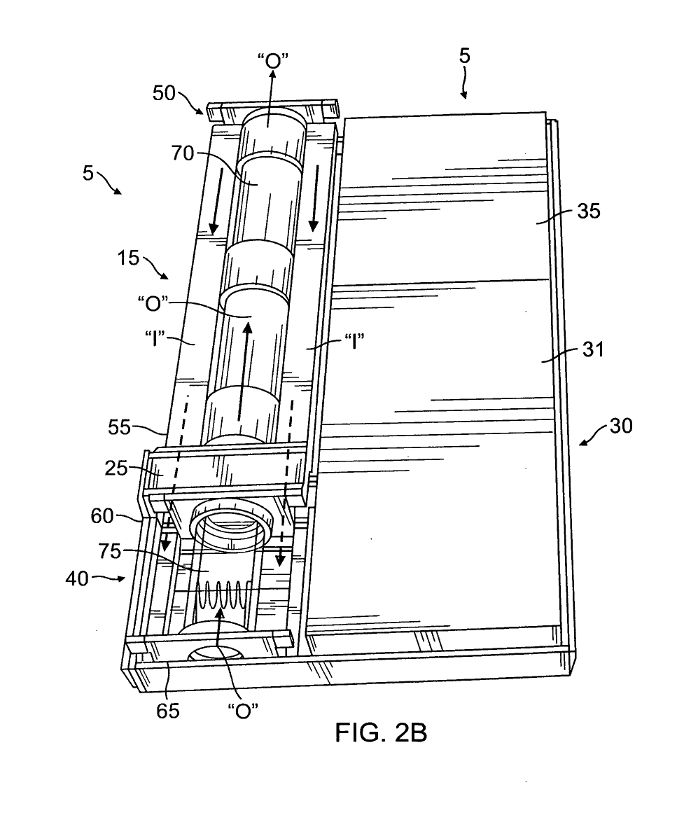

As shown in FIGS. 2A, 2B, and 3, in one embodiment, the vaporizer 5 is configured to provide bidirectional air flow within the vaporizer 4. More specifically, the elongated vapor chamber 15 is configured to receive air from outside the vaporizer 5 at a first end 50 of the vapor chamber 15. The air flows inside the vapor chamber 15 along the inside periphery of the vapor chamber 15 where the air exits a second end 55 of the vapor chamber 15 opposite the first end 50 of the vapor chamber 15, passes through the periphery of the cartridge 25 at intake openings 80 (see directional arrows along periphery of cartridge in FIG. 5), and enters the top end 60 of heating chamber 40.

In this regard, in one embodiment shown in FIG. 3, the heating chamber 40 is constructed of quartz tubing, ceramic, or other suitable heat resistant material and is disposed adjacent the cartridge 25 to receive the flow of air into the periphery of the heating chamber 40 at the top end 60 of the heating chamber 40. The air continues to flow along the periphery of the heating chamber 40 until the air reaches a bottom end 65 of the heating chamber 40. As such, the entrance or input pathway, designated as arrows labeled “I”, for the bidirectional flow of air includes the periphery area of the vapor chamber 15, the cartridge 25, and the heating chamber 40.

Once the air reaches the bottom end 65 of the heating chamber 40 the air flows into a central cavity 70 that extends continuously from the bottom end 65 of the heating chamber 40 to the first end 50 of the vapor chamber 15. As such, the exit or output pathway for the bidirectional flow of air includes the central cavity 70 formed within the heating chamber 40, the cartridge 25, and the vapor chamber 15. Accordingly, the input airflow pathway and the output airflow pathway (bidirectional flow of air) are parallel to each other. The portion of the heating chamber 40 that includes the central cavity 70 is the heating core 75 (see FIG. 2B).

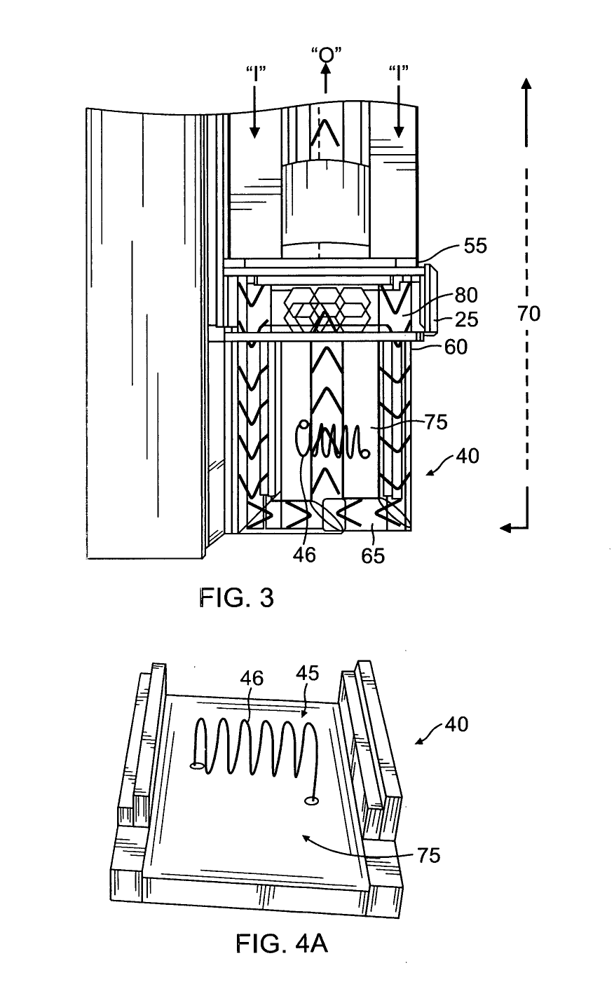

As the air flows into the heating core 75 of the heating chamber 40 the air passes over a heating element 45 such as a coil 46 positioned within the heating core 75 at approximately the center of the heating core 75. In one embodiment, the heating chamber 40 including heating core 75, shown in FIGS. 4A and 4B, is formed by two identical pieces of ceramic, quartz, or other suitable heat resistant material (only one piece is shown in FIG. 4A) that are fitted together and sealed with a sealant such as a food grade sealant. The material of the heating chamber 40 serves as an insulator to protect the vaporizer 5 from high temperature of the coil 46 and to focus the heat within the heating core 75.

In one embodiment, the heating chamber 40 includes sidewalls that are approximately 2 mm thick to form and an internal hollow space that defines the heating core 75 having a volume of approximately 2.1 cubic centimeters (14 mm×6 mm). The coil 46 is heated by the power source 30 to an operating temperature of between approximately 700-800 degrees Fahrenheit in approximately 2 seconds. The temperature of the coil 46 and time it takes to reach the operating temperature is determined by the length of the coil 46 and the power placed on the coil 46 by the power source 30. As shown below, in one embodiment, a single coil is utilized having an approximate length of 105 mm (4.1 inches) when laid out straight and measured from end-to-end. When the coil is bent and includes a plurality of bent sections the approximate length as the coil is 8.30 mm (0.33 inches) with each bend having a height of approximately 4.262 mm (0.166 inches) and a total coil height of approximately 21.61 mm (0.85 inches).

In one embodiment, the coil 46 is a nichrome wire having a 26 gauge thickness. As such, due the relatively small size of the coil 46 an operating temperature of between approximately 700-800 degrees Fahrenheit is obtained in approximately 2 seconds which is considerably faster than comparable known vaporizers that reach operating temperatures in about 30-40 seconds.

Likewise, cooling down of the coil 46 is relatively fast. The rapid heat build-up and cool down characteristics of the coil 46 prevents excessive heat from remaining within the heating core 75 and minimizes power consumption.

Accordingly, when compared to similar type convection vaporizers, the heating chamber 40, heating core 75, and single coil 46 of the vaporizer 5 described herein provides a relatively simpler, faster, and more efficient vaporizer 5 and method for heating of the heating element and convection heating of a substance. Persons of ordinary skill in the art will understand that the volume of the heating core 75 and/or one or more of the dimensions of the coil 46 may be varied to achieve the operating temperature within the time indicated above.

As the air flows over the coil 46 the air is then heated by convection to a temperature of approximately 400 degrees Fahrenheit. As shown in FIG. 3, the heated air is then passed over a substance, herbs for example, such as tobacco, cannabis, lavender, chamomile, and many other types of plant material disposed within the cartridge 25 and positioned within the central cavity 70 to vaporize the substance. Generally, the substance is heated at a rate determined inversely to the rate of airflow drawn through the central cavity 70 by the user. The vaporization of the substance typically results in a vapor containing an active ingredient of the substance.

The vapor containing a product of the substance disposed in the cartridge 25 is drawn up the central cavity 70 and out of the first end 50 of the vapor chamber 15 (see FIG. 2B). The bidirectional pathway for the input “I” of air flow into the vaporizer 5 along the periphery and the output of air flow, designated as arrows labeled “O”, out of the vaporizer 5 within the central cavity 70 is facilitated by the inhalation of a user providing a seal on the central cavity 70 at the first end 50 of the vapor chamber 15 and inhaling.

The heat of the vapor drawn up through that portion of the central cavity 70 beyond the cartridge 25 (output tube) is transferred by way of convection to metal fins (not shown), acting as a heat sink, that are positioned and spaced along the length of the periphery of the vapor chamber 15, thus cooling the vapor that exits the first end 50 of the vapor chamber 15. The heat once removed from the vapor and transferred to the fins is then again transferred by convection to the air being drawn into the vaporizer 5 at the first side 50 of the vapor chamber 15 along the periphery.

The cartridge 25 shown in FIG. 5 is removable and interchangeable with other similar type cartridges. As indicated above, the cartridge 25 is configured to contain the herbal material (substance) upon a mesh screen, provide a point for vaporization of the material, and storage of the remaining byproduct after vaporization of the herbal material. The material remaining after vaporization may be removed to facilitate the addition of subsequent herbal material into the cartridge 25.

The vapor chamber case 16 provides a means for holding the cartridge 25 as well as the output tube portion of the central cavity 70. When the cartridge 25 is removed from the first sidewall portion 11 and the second sidewall portion 12 of the vaporizer 5 any parts containing the material vaporized or any byproducts or residues from vaporizing is likewise removed. This changes the overall shape of the vaporizer 5 in a way that allows for restrictive return packaging to prevent said substances from being shipped with the vaporizer 5.

The vaporizer 5 described herein may further include a power on/off control such as a button or switch, as well as one or more LED indicators for visual verification of the power and battery status. Persons of ordinary skill in the art will understand that the dimensions of the case 10 and placement of the power control and LED may be varied within the spirit and scope of subject matter described herein.

The apparatus and methods of the present invention have been described with some particularity, but the specific designs, constructions and steps disclosed are not to be taken as delimiting of the invention. Obvious modifications will make themselves apparent to those of ordinary skill in the art, all of which will not depart from the essence of the invention and all such changes and modifications are intended to be encompassed within the appended claims.

What is claimed is:

1. A vaporizer for vaporizing a substance, the vaporizing comprising:
a vapor chamber;
a heating chamber; and
a cartridge positioned between the vapor chamber and the heating chamber,
wherein the vapor chamber, the cartridge, and the heating chamber form a continuous bidirectional pathway for airflow within the vaporizer,
wherein the cartridge is configured to contain the substance to be vaporized, and
wherein the heating chamber includes a heating core having a heating element disposed therein for convection heating of a portion of the airflow that vaporizes the substance contained in cartridge.

2. The vaporizer of claim 1, further includes a central cavity extending within the vapor chamber, the cartridge, and the heating chamber to form a exit pathway for the airflow, the entrance pathway for the airflow formed by a periphery area of the vapor chamber, the cartridge, and the heating chamber.

3. The vaporizer of claim 2, wherein the heating core of the heating chamber forms a portion of the central cavity.

4. The vaporizer of claim 1, wherein the heating element is a nichrome wire.

5. The vaporizer of claim 4, wherein the nichrome wire has a 26 gauge thickness.

6. The vaporizer of claim 5, wherein the nichrome wire having a 26 gauge thickness is positioned in approximately the center of the heating core.

7. The vaporizer of claim 1, further includes a power source and associated circuitry connected to the heating element to supply power to the heating element,
wherein the heating element is configured to reach a temperature of between approximately 700-800 degrees Fahrenheit when power is supplied to the heating element from the power source.

8. The vaporizer of claim 7, wherein the power source is two rechargeable 3.7 volt lithium polymer batteries wired in series.

9. A heating chamber for heating a portion of airflow within a vaporizer comprising:
a heating core formed in the center of the heating chamber;
a heating element disposed within the heating core for convection heating of a portion of an output airflow within the vaporizer.

10. The heating chamber of claim 9, wherein the heating chamber further includes an intake opening positioned at the periphery of the heating chamber to receive a portion of an input airflow.

11. The heating chamber of claim 9, wherein the heating chamber is constructed of ceramic, the heating core has an approximate volume of 2.1 cubic centimeters, and
wherein the heating element is a nichrome wire positioned in the approximate center of the heating core.

12. The heating chamber of claim 9, where the heating element is a nichrome wire.

13. The heating chamber of claim 12, wherein the nichrome wire that includes a plurality of bent sections each having a height of approximately 4.262 mm, a total coil height of approximately 21.61 mm, and an approximate coil length of 8.30 mm.

14. The heating chamber of claim 13, wherein the nichrome wire reaches an operating temperature of between approximately 700-800 degrees Fahrenheit.

15. The heating chamber of claim 12, wherein the nichrome wire has a 26 gauge thickness.

16. The heating chamber of claim 15, wherein the nichrome wire is positioned in approximately the center of the heating core.

17. The heating chamber of claim 16, wherein the nichrome wire reaches an operating temperature of between approximately 700-800 degrees Fahrenheit.

18. A bidirectional airflow vaporizer for vaporizing a substance, the vaporizing comprising:
a heating chamber;
wherein the heating chamber includes a central hollow heating core having a single coil disposed therein for convection heating of an output portion of the airflow that vaporizes the substance.

19. The vaporizer of claim 18, wherein the heating chamber further includes an intake opening positioned at the periphery of the heating chamber to receive a portion of an input airflow.

20. The vaporizer of claim 18, wherein the single coil is a nichrome wire positioned in approximately the center of the heating core and reaches an operating temperature of between approximately 700-800 degrees Fahrenheit.
